(19) United States
(12) Patent Application Publication
(10) Pub. No.: US 2005/0049625 A1
(43) Pub. Date: Mar. 3, 2005

(54) Device and method for intradermal cell implantation

(76) Inventors: Steven Shaya, Highlands, NJ (US); Peter Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/925,518

(22) Filed: Aug. 24, 2004

Related U.S. Application Data
(60) Provisional application No. 60/498,143, filed on Aug. 26, 2003.

(51) Int. Cl. A61B 17/34

(52) U.S. Cl. 606/187

(57) ABSTRACT

The present invention relates to a device and method for implanting hair follicle-inducing cells within the scalp. The hair follicle cells are generated by the use of tissue culture process from autologous donor cells. The cells are placed upon a microprojection array for implantation within the scalp utilizing predetermined parameters of angle, density and depth.

### CROSS REFERENCE TO RELATED APPLICTAIONS

This application claims the benefit of U.S. Provisional Application No. 60/498,143, filed Aug. 26, 2003.

### FIELD OF THE PRESENT INVENTION

This invention relates to administering and implanting viable cells into the tissue of a patient. More particularly, the invention relates to a cell implantation system for transdermally delivering viable cells through one or more cellular layers of the integument surrounding the organism using skin-piercing microprojections that are preferably adapted to retain the cells.

### BACKGROUND OF THE INVENTION

The loss of hair (alopecia) due to age and or disease is a common occurrence. Various methods of correcting this problem have been tried. Such methods as wigs, toupees, weaves, and even spray-on-hair have been utilized. These methods attempt to address the problem by cosmetically hiding or masking the hair loss rather than halting or reversing the physiological changes that resulted in the hair loss itself.

Prior art attempts to correct or reverse hair loss have utilized pharmaceuticals. A notable example is minoxidil (Rogaine®) which is usually applied topically to the scalp. Finasteride (Propecia®) is another pharmaceutical intended to correct hair loss. It is used as an orally delivered systemic medication. These pharmaceutical approaches have proven variable in their efficacy of reversing hair loss, and do not work for a substantial percentage of patients.

In contrast to pharmaceutical approaches, hair follicle transplants have provided greater success. Such methods entail the autologous procedure of the surgical removal of hair follicles or clusters of hair follicles from regions of the scalp containing viable follicles and transplanting them to other regions of the scalp which have sustained hair loss.

Early procedures required the transplantation of fairly large clusters of hair cells. This limitation produced a very artificial look to the resulting hair growth. More advanced surgical techniques have been developed, allowing transplantation of smaller and smaller clusters, thus improving the esthetic quality of the resulting new hair growth.

Nevertheless, these prior art techniques have certain limitations. Chiefly, transplantation of existing follicles does not increase the number of viable follicles, it only relocates them. If the transplant is initiated with a patient who has already sustained significant hair loss, there may not be a sufficient number of follicles to fully restore the entire scalp. Further, the life span transplanted follicles is uncertain. Moreover, the physiological basis for the hair loss has not been corrected. Consequently, the rate at which the follicles will die off may not have been altered as a result of this transplant. Thus, if a patient's condition would inherently lead to total baldness, the transplant procedure may not alter the progression. The transplanted follicles may follow the normal progression of decay in the new location.

Yet another draw back to transplantation procedures is the high level of skill required for this procedure. They need to be performed by trained physicians, under sterile conditions and usually entail multiple sessions of transplantation.

To overcome these limitations, attempts have been made to increase the number of viable follicles. For example, a number of patents have issued which deal with tissue culture growth of hair progenitor cells and the macro scale surgical placement of these cells into the skin. In U.S. Pat. No. 4,919,664 to Oliver, it is disclosed that the placement of cultured dermal papillae cells into the dermis so that the cells are in contact with epidermal cells will result in the formation of fully functioning hair follicles. These cells initiate the differentiation of surrounding cells so that hair follicles are formed. In the referenced procedure, slits were made in the skin that were in the range of 1 to 3 millimeters long and made to a depth greater then the full depth of the epidermis. The cells were introduced in a volume of from 0.5 microliters to 50 microliters containing 1000 to 1,000,000 cells.

In another example, WO 02/060039 to Barrows, a procedure is described in which dermal papillae, a morphological component of hair follicles, were dissected from donor hair follicles and then grown in tissue culture media. In this procedure, a hyaluronate-gelatin matrix was packed inside a needle containing a 0.0035-inch diameter wire. The needle/wire combination was used to scrape confluent cells from the dermal papilla tissue culture flasks. The needle/wire and attached cells were placed in culture media and the cells were allowed to grow for about a week. Then a small blep was created in the scalp at the location that hair growth is desired. The blep was created by injecting a solution of sodium hyaluronate into the scalp. The blep was punctured with a knife and the wire and the attached cells were then inserted into the blep. The needle was removed and then later the wire, leaving the cells positioned within the scalp.

These references indicate that it is possible to culture viable hair follicle-inducing cells and seed them in a patient's scalp. However, these prior art methods require tedious manipulation and a high level of skill, making them relatively impractical as a treatment for hair loss.

Thus, there remains a need for a hair loss treatment that provides a high rate of true hair restoration, requires less skill than the traditional hair transplant techniques, increases the total number of available and viable hair follicles and quickly treats significant portions of the scalp.

Accordingly, it is an object of the invention to provide a method of treating hair loss by transdermally delivering hair follicles using a microprojection array.

### SUMMARY OF THE INVENTION

In accordance with the above objects and those that will be mentioned and will become apparent below, the invention comprises a transdermal delivery system for implanting hair follicles in a patient with a microprojection array having a plurality of stratum corneum-piercing microprojections and a formulation of hair-follicle inducing cells deposited on at least one of the microprojections.

Preferably, the microprojections of the array are configured to retain the formulation. In one embodiment, the cavity is symmetrical and concave. The cavity can also comprise a retention barrier, configured to retain the formulation upon insertion and allow the release of the formulation upon withdrawal of the microprojection from the tissue. Moreover, the cavity can be positioned on the broad face of the microprojection or upon the narrow edge.

In some embodiments, the microprojection also has a pressure conduit configured to communicate pressure to the cavity to facilitate dislodging the formulation.

In another aspect of the invention, the microprojection has an absolute orientation configured to produce a desired wound path. For example, the absolute orientation can be the angle of the microprojection with respect to the sheet, such as about 45 to 90 degrees. The absolute orientation is configured to result in a desired hair follicle orientation.

In one embodiment of the invention, the formulation is selectively applied to a specific portion of the microprojection. For example, the microprojection can have a hydrophobic coating except on the cavity so that the aqueous formulation is retained only in the cavity.

In another embodiment, the formulation is frozen on the microprojection to help retain it during insertion. The formulation can be allowed to thaw once the microprojection is inserted through the stratum corneum, to deliver the hair follicle-inducing cells.

In yet another embodiment, a bioerodible polymer is applied to the microprojection and the formulation is incorporated within the polymer. The array is left in the tissue until the polymer erodes to deliver the hair follicle-inducing cells.

Preferably, the devices and methods of the invention are directed to the implantation of cultured autologous dermal papilla cells. Alternatively, allogeneic cells and xenogeneic cells can also be used.

The method of the invention generally comprises the steps of providing a microprojection array having a plurality of stratum corneum-piercing microprojections and a formulation of hair-follicle inducing cells deposited on at least one of the microprojections, applying the microprojection array to the patient so that the microprojection formulation of hair-follicle inducing cells to tissue beneath the stratum corneum. Preferably, the method comprises retaining a substantial portion of the formulation on the microprojection and releasing the formulation upon withdrawal of the microprojection.

In an embodiment of the invention, the method also comprises selecting an absolute orientation and a relative orientation of the microprojection to create a desired wound path. Preferably, hair follicle orientation is influenced by creating the desired wound path.

The methods of the invention also include growing hair on a patient by transdermally implanting hair follicle-inducing cells with a microprojection array.

Yet another method of the invention is the formation of a hair loss treatment device by providing a microprojection array and depositing a formulation of hair follicle-inducing cells on the microprojection array.

In one aspect of the invention, at least one microprojection has a hydrophobic material except for a non-hydrophobic cavity so that when the formulation is applied to the microprojection, the formulation is retained only on the non-hydrophobic cavity.

In another aspect of the invention, the formulation is frozen on the microprojection array. Alternatively, the formulation can be included in a bioerodible polymer that is applied to at least one of the microprojections of the array.

### BRIEF DESCRIPTION OF THE DRAWINGS

FIG. 1A is a perspective view of a portion of one embodiment of the invention, showing a microprojection array having cavities on some of the microprojections which would retain formulations, suspensions or coatings of the viable elements;

FIG. 1B is section view of one microprojection showing one embodiment of a cavity for retaining a formulation of hair follicle-inducing cells;

FIG. 2A is a perspective view of a single microprojection showing alternate embodiments of two cavities on the microprojection for containing a suspension of the viable elements;

FIG. 2B shows a sectional view of the microprojection shown in FIG. 2A;

FIG. 3 is a perspective view of a single microprojection containing another embodiment of a cavity positioned on an edge of the microprojection and an optional pressure conduit for enabling the release of the suspension into the surrounding tissue; and

FIG. 4 is a perspective view of a single microprojection similar to the one shown in FIG. 3, but including an additional retention pocket.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a microprojection” includes two or more such microprojections and the like.

### Definitions

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human. In one embodiment of the invention, the microprojections have a projection length less than 1000 microns. In a further embodiment, the microprojections have a projection length of less than 500 microns, more preferably, less than 250 microns. The microprojections typically have a width and thickness of about 5 to 50 microns. The microprojections can also have a width of about 75 to 500 microns. The microprojections can be formed in different shapes, such as needles, hollow needles, blades, pins, punches, and combinations thereof. As such, the terms “microprojections,” “microprotrusions,” “microblades” and “microneedles” are used throughout interchangeably.

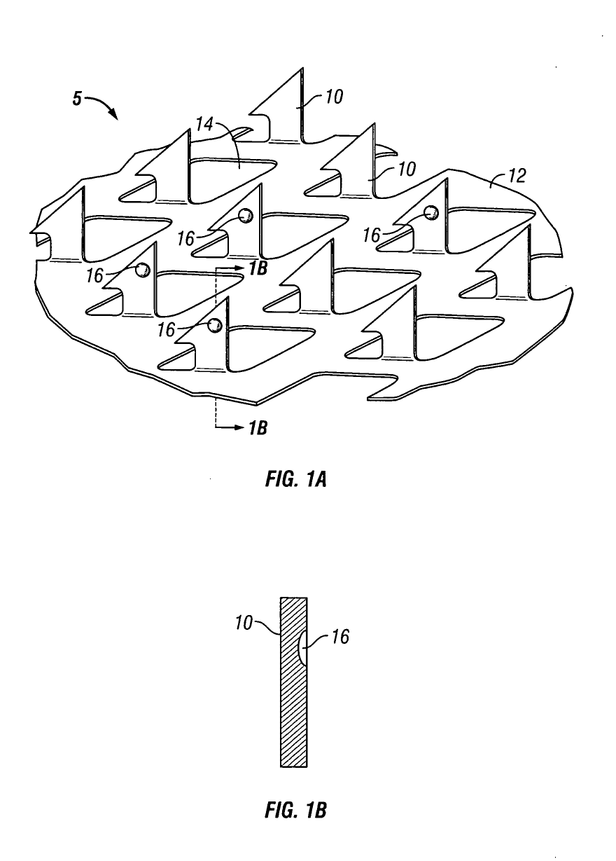

The terms “delivery member,” “microprojection array” and “microprojection member”, as used herein, generally connote a plurality of microprojections arranged in an array for piercing the stratum corneum. The array can be formed by etching or punching a plurality of microprojections from a thin sheet and folding or bending the microprojections out of the plane of the sheet to form a configuration such as that shown in FIG. 1 and described in Trautman, et al., U.S. Pat. No. 6,083,196, which is hereby incorporated by reference in its entirety. References to the area of the sheet or member and reference to some property per area of the sheet or member, are referring to the area bounded by the outer circumference or border of the sheet.

The microprojection array may also be formed in other known manners, such as by forming one or more strips having microprojections along an edge of each of the strip(s) as disclosed in Zuck, U.S. Pat. No. 6,050,988, which is hereby incorporated by reference in its entirety. Other microprojection members that can be employed with the present invention include, but are not limited to, the members disclosed in U.S. Pat. Nos. 6,083,196, 6,050,988, 6,091,975, 5,879,326 and 5,983,136; which are incorporated by reference herein in their entirety.

The present invention relies upon the use of tissue culture techniques to grow dermal papilla cells that are harvested from the patient. Alternatively, cells obtained from allogeneic sources or manipulated xenogeneic species could be used. These cells are then implanted into the scalp of the same patient by the use of a microprojection array. The cells are implanted so as to be in contact with the epidermis. This results in the formation of fully functional hair follicles. A key advantage of this technique is that it provides an increase in the number of hair follicles available to the patient, provided there are sufficient viable follicles still remaining on the scalp that can be harvested and used to initiate the tissue culture procedures.

Delivery of the cultured cells is accomplished when the microprojections pierce the outer integument, such as the stratum corneum, of the organism and the microprojections deposit the cells at the appropriate location within or below the integument of the individual. Specifically, the invention relates to a device and method that will implant hair-follicle inducing cells at predetermined locations within the scalp for the purpose of causing the development of hairs follicles and the subsequent growth of new hair

Thus, an aspect of the invention is the use of a small array of microprojections. The microprojections are designed to hold and retain hair follicle progenitor cells that have been grown by tissue culture techniques. This array is loaded with the progenitor cells on the microprojections and then applied to the scalp. The microprojections pierce the scalp and deposit the cells. The array contains microprojections of the appropriate lengths, orientations and spacing in order to effectuate proper placement of the cells, in terms of spacing over the surface of the scalp as well, the depth in the epidermis of the scalp at which the cells are deposited and the likely orientation of resulting hair follicles.

Microprojection arrays offer a number of advantages in the practice of the invention. Arrays can be created with microprojections having varying lengths to control penetrations, with varying densities of microprojections per area to control the number of hair-follicle inducing cells delivered and with microprojections having varying angles to control the angle of entrance of the microprojection into the scalp to dictate the angle that the resultant hair fiber makes with the scalp. As one having skill in the art will appreciate, the angle of entrance is a critical factor in obtaining a natural looking hair pattern.

Generally, one aspect of the present invention comprises a microprojection array, a plate or sheet from which a series of microprojections extend, typically at an angle ranging from 45 to 90 degrees from the plate. Preferably, the length of each microprojection can physically be in the range of 100 to 600 micrometers. The plate, typically made of metal, preferably titanium, range in overall area up to 10 cm2. The concentration of microprojections can range between 10 to 1,000 microprojections per cm2. Within these physical limitations, all of the above parameters can be varied in order to provide an implantation regimen to suit the needs of a particular patient.

Referring now to FIG. 1A, one embodiment of a microprojection array 5 is illustrated for use with the present invention. FIG. 1A shows a portion of microprojection array 5 having a plurality of microprojections 10, which are configured to pierce or cut a biological surface, such as the stratum corneum. In this embodiment, the microprojections are formed by etching or punching a plurality of microprojections 10 from a thin metal sheet 12. As shown, microprojections 10 are bent out of the plane of the sheet to substantially a 90° angle from sheet 12, partially forming openings 14. Sheet 12 may be incorporated into a delivery patch including a backing for sheet 12 (not shown) and may additionally include adhesive for adhering the patch to the skin.

Metals such as stainless steel and titanium are preferred. Metal microprojection members are disclosed in Trautman et al, U.S. Pat. No. 6,083,196; Zuck, U.S. Pat. No. 6,050,988; and Daddona et al., U.S. Pat. No. 6,091,975; the disclosures of which are fully incorporated herein by reference. Other microprojection members that can be used with the present invention are formed by etching silicon using silicon chip etching techniques or by molding plastic using etched micro-molds. Silicon and plastic microprojection members are disclosed in Godshall et al., U.S. Pat. No. 5,879,326, the disclosure of which is fully incorporated herein by reference.

Also shown in FIG. 1A are one or more cavities 16, located on one of the broad surfaces 15a of one or more of microprojections 10. These and other cavities are designed to hold a suspension, either liquid or frozen of hair-follicle inducing cells. The hair-follicle inducing cells can also be contained within a bioerodible polymer, which is deposited within the cavities.

FIG. 1B is a sectional view of one of the microprojections 10. Cavity 16 is shown in FIGS. 1A and 1B as being circular, but any number of other shapes are possible, some of which are disclosed below.

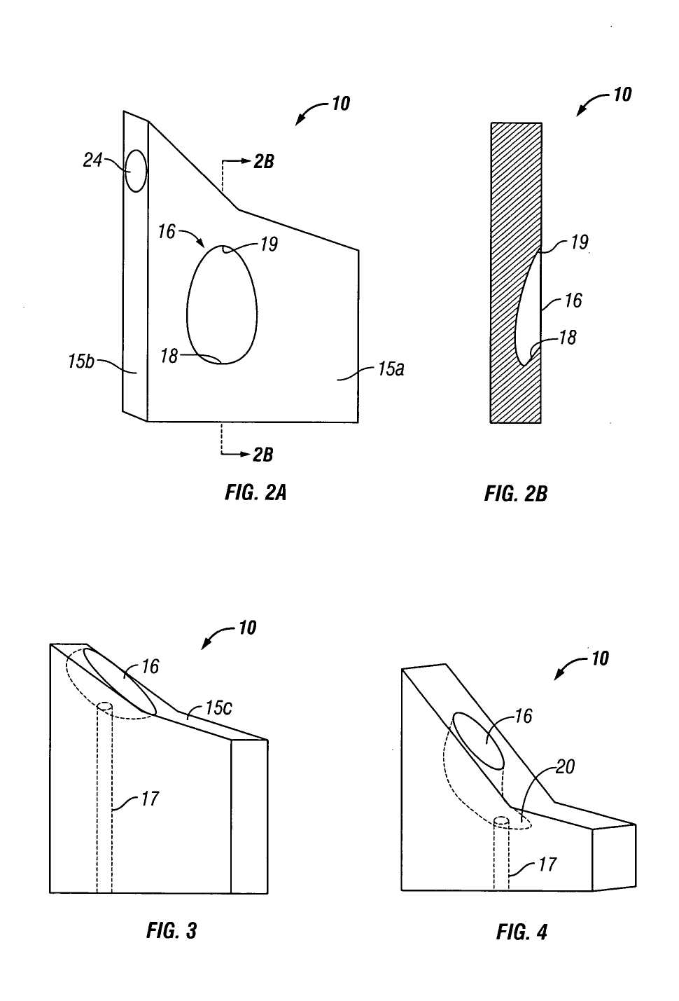

FIG. 2A illustrates a single microprojection 10. Microprojection 10, because it must be able to pierce the biological surface, usually has a flat blade-like shape, with a broad surface 15a and a narrow edge 15b. Cavity 16 is shown located along one of the broad surfaces 15a of microprojection 10. Cavity 16 can be a symmetrical concave cavity as shown in FIGS. 1A and 1B. However, as shown in FIGS. 2A and 2B, cavity 16 can also be non-symmetrical and adapted to have a retention barrier 18.

Preferably, cavity 16 is configured so that upon insertion of microprojection array 5 into the scalp, the formulation contained in cavity 16 is substantially trapped and/or caught behind retention barrier 18. This helps prevent the formulation from being dislodged during insertion. Further, the leading edge 19 of cavity 16 is preferably gently sloped, so that withdrawal of microprojection array 5 from the scalp allows the formulation to slide easily out of cavity 16. Thus, the formulation is deposited within the scalp tissue at the depth that cavity 16 was positioned after insertion of microprojection array 5.

The narrow edge 15b may also be the site of a cavity, such as cavity 24. Cavity 24 can have, but is not limited to, the same symmetrical configuration as shown in FIGS. 1A and 11B, but could also have the configuration of cavity 16 as shown in FIGS. 2A and 2B, or the configuration shown in FIGS. 3 and 4.

FIG. 3 shows cavity 16 located on the leading edge 15c. A cavity located in this position has the advantage that the insertion process tends to force the formulation or suspension located within cavity 16 down into the cavity.

An optional pressure conduit 17 can be included to allow exertion of pressure along the conduit to aid in the depositing of the formulation or suspension within the tissue. Preferably, pressure conduit 17 can be used to conduct either liquid or gas pressure to one or more of the microprojections. After the microprojection array 5 has been inserted into the biological surface, a brief application of gas or liquid pressure gently urges the formulation out of cavity 16. Though the pressure conduit is only shown in FIGS. 3 and 4, it could be included in the embodiments shown in FIGS. 1A, 1B, 2A or 2B.

FIG. 4 shows an embodiment similar to that shown in FIG. 3, but which also includes a retention pocket 20 located in the lower portion of cavity 16. The addition of the retention pocket further aids in the retention of the formulation during insertion. The inclusion of the pressure conduit 17, as described previously aids in release or deposition of the formulation within the tissue after the proper insertion of the microprojection array 5.

As discussed above, the microprojections are designed to contain cavities or other means of retaining hair follicle inducing cells. The microprojection array, with the hair follicle inducing cells disposed within the cavities of the microprojections, is then inserted into the tissue on the scalp. Though the primary discussion herein focuses on implanting hair-follicle inducing cells into the scalp, it should be understood that the device and methods of the invention can be applied to any area of the body on which hair is desired to be grown, such as the eyebrows, face or arms.

Another aspect of the invention is directed to producing a desirable orientation of the implanted hair follicles. The delivery of follicle inducing cells into the scalp leaves a wound for each entry path. The path of the wounding, or wound path, in the epidermis during the insertion and withdrawal of the microprojection array can influence the direction of follicle development and the orientation that the resulting hair fiber takes with respect to the implanted tissue. Accordingly, the microprojection array can be configured to control the orientation of the entry and withdrawal path of the microprojections.

Two aspects of the wound path bear upon the appearance of hair follicle transplantation. The first is the absolute angle that the path makes with respect to the surface of the tissue. The second is the relative orientation of the wound path with the respect to the whole patient. For example, if an implantation is to be made directly on the top of the head at an absolute orientation of about 45 degrees with respect to the scalp surface, then the relative orientation can be chosen to produce a desired pattern. For example, if the relative orientation is directed towards the back of the head, then the developing hair shaft generally will be directed backwards. Correspondingly, if the angle of the wound is oriented towards the front of the head, then the developing hair shaft generally will be directed forwards. Thus, the absolute orientation and the relative orientation angles can be selected to achieve an aesthetically pleasing pattern of hair growth.

To control the absolute orientation, microprojections 10 are positioned at the desired angle relative to the plane of array 5. The array can then be applied to the scalp in the desired relative orientation. Preferably, any absolute orientation of microprojections 10 on array 5 is marked to ensure proper relative placement. This can be accomplished in any suitable manner. For example, one edge of the base of the microprojection array could be notched or in some other manner marked, to indicate the direction angle of the microprojections.

In some embodiments, microprojection array 10 is preferably suspended in a retainer ring as described in detail in Co-Pending U.S. patent application Ser. No. 09/976,762, filed Oct. 12, 2001, which is incorporated by reference herein in its entirety. After placement of the microprojection array 10 in the retainer ring, the microprojection array 10 is applied to the patient's scalp, preferably with an impact applicator, such as disclosed in Co-Pending U.S. patent application Ser. No. 09/976,798, filed Oct. 12, 2001, which is incorporated by reference herein in its entirety. The microprojection array is attached to the ring by frangible tabs. When the stored energy within the applicator is released, a piston is driven onto the microprojection array, breaking the frangible tabs, releasing the microprojection array from the mounting ring and driving it into the skin. The ring can be marked to indicate the direction of angles of the microprojections mounted therein to facilitate proper relative orientation upon insertion into the scalp.

With both the angle of the microprojections and the orientation properly marked or otherwise indicated, the operator is able to align the orientation of the microprojection in the desired direction with respect to the body of the recipient. For example, it may be desirable to orient the hair follicles perpendicular to a natural part in the hair, radially around the crown, or perpendicular to the long axis of the arm.

In one embodiment, the microprojection array is configured to be mounted in a handle. Preferably, the array should fit in only one orientation. Both the handle and the microprojection array can be adapted so that once assembled, an indicator mark on the handle allows the operator to apply the microprojection array with the proper orientation.

Another aspect of the invention is directed to the deposition of hair follicle formulations within cavities 16 of microprojections 10 to effectuate good hair growth. In one embodiment, all areas of the microprojections 10 except cavities 16 are coated with a hydrophobic material. Then the microprojection array is then dipped into an evenly dispersed aqueous suspension of cell clusters. The hydrophobic material repels the aqueous suspension, allowing only the non-hydrophobic cavities to be coated. Suitable methods of coating microprojections and apparatus useful to apply such coatings are disclosed in U.S. patent application Ser. Nos. 10/045,842, filed Oct. 26, 2001, Ser. No. 10/099,604, filed Mar. 15, 2002, 60/484,142, filed Jun. 30, 2003, and 60/285,576; the disclosures of which are incorporated by reference herein.

In another embodiment, the cell suspensions are deposited within the cavities of the microprojection array and then frozen. In this method, each array is used only once. Because of the relatively small volumes involved, the frozen suspensions, thaw to a liquid state very quickly once placed within the scalp tissue to allow deposition of the cell clusters.

In yet another embodiment, the cell clusters are incorporated within a bioerodible polymer that is then coated directly on the microprojections or deposited within one or more cavities on one or more faces of the microprojections. The microprojection array is then be inserted into the scalp and left in place long enough for the polymer to erode and release the cell clusters into the scalp tissue.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A transdermal delivery system for implanting hair follicles in a patient comprising a microprojection array having a plurality of stratum corneum-piercing microprojections and a formulation of hair-follicle inducing cells deposited on at least one of said microprojections.

2. The transdermal delivery system of claim 1, wherein said at least one microprojection has a cavity and said formulation of hair-follicle inducing cells is deposited within said cavity.

3. The transdermal delivery system of claim 2, wherein said hair-follicle inducing cells comprise dermal papilla cells cultured from the group consisting of autologous, allogeneic, and xenogeneic primary sources.

4. The transdermal delivery system of claim 2, wherein said cavity has a symmetrical concave configuration.

5. The transdermal delivery system of claim 2, wherein said cavity further comprises a retention barrier.

6. The transdermal delivery system of claim 5, wherein said retention barrier is configured to substantially retain said deposited formulation upon insertion of said microprojection into tissue and to release said deposited formulation upon withdrawal of said microprojection from tissue.

7. The transdermal delivery system of claim 6, wherein said cavity further comprises a sloped leading edge configured to facilitate release of said deposited formulation upon withdrawal of said microprojection from tissue.

8. The transdermal delivery system of claim 2, wherein said at least one microprojection has a broad surface and a narrow edge.

9. The transdermal delivery system of claim 8, wherein said cavity is positioned on said broad surface.

10. The transdermal delivery system of claim 9, wherein said cavity is positioned on said narrow edge.

11. The transdermal delivery system of claim 10, wherein said at least one microprojection further comprises a pressure conduit configured to communicate pressure to said cavity to facilitate dislodging said deposited formulation.

12. The transdermal delivery system of claim 10, wherein said cavity further comprises a retention pocket that is configured to substantially retain said deposited formulation upon insertion of said microprojection into tissue and to release said deposited formulation upon withdrawal of said microprojection from tissue.

13. The transdermal delivery system of claim 2, wherein said cavity is configured to substantially retain said deposited formulation upon insertion of said microprojection into tissue.

14. The transdermal delivery system of claim 1, wherein said at least one microprojection has an absolute orientation configured to produce a desired wound path.

15. The transdermal delivery system of claim 14, wherein said delivery system further comprises a sheet from which said at least one microprojection protrudes and said absolute orientation comprises an angle of said at least one microprojection with respect to said sheet.

16. The transdermal delivery system of claim 14, wherein said absolute orientation is configured to result in a desired hair follicle orientation.

17. The transdermal delivery system of claim 15, wherein said angle is between about 45 degrees and 90 degrees.

18. The transdermal delivery system of claim 2, wherein said at least one microprojection has a hydrophobic coating except on said cavity and wherein said formulation is aqueous.

19. The transdermal delivery system of claim 1, wherein said formulation is frozen.

20. The transdermal delivery system of claim 1, further comprising a bioerodible polymer applied to said at least one microprojection and wherein said formulation is incorporated within said bioerodible polymer.

21. The transdermal delivery system of claim 1, wherein said formulation comprises an autologous tissue culture of dermal papilla cells.

22. The transdermal delivery system of claim 1, wherein said formulation is selected from the group consisting of allogeneic cells and xenogeneic cells.

23. A method for treating hair loss in a patient, comprising the steps of:
providing a microprojection array having a plurality of stratum corneum-piercing microprojections and a formulation of hair-follicle inducing cells deposited on at least one of said microprojections;
applying said microprojection array to said patient so that said at least one microprojection pierces the stratum corneum of said patient; and
delivering said formulation of hair-follicle inducing cells to tissue beneath the stratum corneum.

24. The method of claim 22, wherein said step of applying said microprojection array comprises inserting said at least one microprojection through said stratum corneum so that a substantial portion of said formulation remains deposited on said microprojection.

25. The method of claim 23, wherein the step of delivering said formulation comprises withdrawing said at least one microprojection so that a substantial portion of said formulation remains in tissue beneath the stratum corneum.

26. The method of claim 22, further comprising the step of freezing the formulation on said at least one microprojection and wherein the step of delivering said formulation comprises allowing said formulation to thaw after said at least one microprojection pierces the stratum corneum.

27. The method of claim 22, wherein said microprojection array further comprises a bioerodible polymer applied to said at least one microprojection and wherein said formulation is incorporated within said bioerodible polymer and wherein said step of delivering said formulation comprises allowing said bioerodible polymer to dissolve within tissue below the stratum corneum.

28. The method of claim 22, further comprising the step of selecting an absolute orientation and a relative orientation of said at least one microprojection to create a desired wound path.

29. The method of claim 27, further comprising the step of influencing hair follicle orientation by creating said desired wound path.

30. A method for growing hair on a patient comprising the step of transdermally implanting hair follicle-inducing cells with a microprojection array.

31. A method for forming a hair loss treatment device, comprising the steps of:
providing a microprojection array; and
depositing a formulation of hair follicle-inducing cells on said microprojection array.

32. The method of claim 30, wherein said step of providing a microprojection array comprises providing an array having at least one microprojection with a hydrophobic coating and a non-hydrophobic cavity and wherein said step of depositing said formulation comprises applying said formulation to said at least one microprojection whereby said formulation is retained on said non-hydrophobic cavity and not retained on said hydrophobic coating.

33. The method of claim 30, further including the step of freezing said deposited formulation after said formulation is deposited.

34. The method of claim 30, wherein the said of depositing said formulation on said at least one microprojection comprises applying a bioerodible polymer to said at least one microprojection and incorporating said formulation within said bioerodible polymer.
